(19) United States
(12) Patent Application Publication
(10) Pub. No.: US 2024/0075190 A1
(43) Pub. Date: Mar. 7, 2024

(54) IODINATED CROSSLINKED HYDROGELS AND METHODS OF FORMING THE SAME

(71) Applicant: Boston Scientific Scimed, Inc., Maple Grove, MN (US)

(72) Inventors: Yen-Hao Hsu, Shrewsbury, MA (US); Joseph Thomas Delaney, Jr., Minneapolis, MN (US); Cristian Parisi, Boston, MA (US)

(73) Assignee: Boston Scientific Scimed, Inc., Maple Grove, MN (US)

(21) Appl. No.: 18/449,907

(22) Filed: Aug. 15, 2023

Related U.S. Application Data
(60) Provisional application No. 63/398,765, filed on Aug. 17, 2022.

(51) Int. Cl. A61L 27/52; C08L 79/02

(52) U.S. Cl. CPC A61L 27/52; C08L 79/02; C08L 2203/02; C08L 2312/00

(57) ABSTRACT

In some aspects, the present disclosure pertains to systems for forming a hydrogel that comprise (a) an iodinated polyamino compound and (b) a reactive multi-arm polymer that comprises a plurality of hydrophilic polymer arms having unsaturated end groups that are reactive with amino groups of the iodinated polyamino compound. Other aspects of the present disclosure pertain to medical hydrogels that are formed by crosslinking the iodinated polyamino compound and the reactive multi-arm polymer of such systems. Further aspects of the present disclosure pertain to medical procedures that can be performed using such systems.

### CROSS-REFERENCE TO RELATED APPLICATIONS

This application claims the benefit of U.S. Provisional Patent Application Ser. No. 63/398,765 filed on Aug. 17, 2022, the disclosure of which is incorporated herein by reference.

### FIELD

The present disclosure relates to iodinated crosslinked hydrogels and to methods of making and using iodinated compounds and hydrogels, among other aspects. The iodinated crosslinked hydrogels of the present disclosure are useful, for example, in various biomedical applications.

### BACKGROUND

In vivo crosslinked hydrogels based on (star-poly(ethylene glycol) (star-PEG) polymers functionalized with reactive ester end groups which are reacted with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR® Clinical Trial Results,” Imaging Technology News, Oct. 27, 2016.

Hydrogels in which some of the star-PEG branches have been functionalized with 2,3,5-triiiodobenzamide (TIB) groups replacing part of ester end groups, such SpaceOAR® Vue, have also been developed, which provide enhanced radiocontrast. See “Augmenix Receives FDA Clearance to Market its TraceIT® Tissue Marker,” BusinessWire Jan. 28, 2013. TraceIT® hydrogel remains stable and visible in tissue for three months, long enough for radiotherapy, after which it is absorbed and cleared from the body. Id.

While the above approach is effectual, some issues arise as a result of incorporation the functional group, TIB. First, the TIB presents poor water solubility, and this characteristic influences how many of TIB groups can be added to the PEG arms before it impacts the ability to form a smooth, consistent hydrogel. Moreover, the overall functionalization process requires multiple steps from commercially available hydroxyl-terminated 8-arm PEG to its functionalized form with two different end groups (i.e., TIB and succinimidyl glutarate (SG) groups). This adds complexity to the star-PEG manufacturing process, which results in a dramatic increase in product cost and product quality control difficulties. Lastly, using the arms of the star polymer to functionalize the hydrogel with iodine means that there are fewer arms available to crosslink. This can be overcome by adding more polymer, but the loading of solids increases, which can adversely impact viscosity. Reducing the molecular weight can cut down on the loading of solids, but this also results in a lower melting point, which has a large impact on storage and shipping conditions, and introduces problems with processability. An additional effect of the reduced crosslink density per star polymer is that the resulting gel has a slower cure rate, which means the gel is liquid and mobile in vivo for longer time periods, opening up opportunities for unintended side-reactions and material displacement. Finally, star PEG labeled with TIB end groups often show discoloration from thermal degradation. While this doesn't impact their functionality, this is a cosmetic defect that is preferably avoided. For these reasons, an innovative strategy for iodine-labeled crosslinkable hydrogels is desired.

The present disclosure is directed to an alternative approach to that above. Rather than using the arms of the star polymer to functionalize the hydrogel with iodine, in the present disclosure the crosslinker for the star polymer is functionalized with iodine. Moreover, the use of reactive succinimidyl glutarate groups on the star polymer may be avoided.

### SUMMARY

In some aspects, the present disclosure pertains to systems for forming a hydrogel that comprise (a) an iodinated polyamino compound and (b) a reactive multi-arm polymer that comprises a plurality of hydrophilic polymer arms having unsaturated end groups that are reactive with amino groups of the iodinated polyamino compound.

In some of these embodiments, the iodinated polyamino compound comprises a polyamino moiety that is linked to a carboxy-substituted iodinated moiety by an amide group. In some of these embodiments, the carboxy-substituted iodinated moiety comprises an iodinated group, such as an iodinated aromatic group, and a carboxylic acid or carboxylate group. In some of these embodiments, the carboxy-substituted iodinated moiety is an iodinated amino acid residue. In some of these embodiments, the polyamino moiety comprises a plurality of —(CH₂)ₓ—NH₂ groups where x is 0, 1, 2 3, 4, 5 or 6. In some of these embodiments, the polyamino moiety comprises two or more amino acid residues selected from residues of lysine, ornithine, and combinations thereof.

In some embodiments, which can be used in conjunction with the above aspects and embodiments, the unsaturated end groups are selected from acrylate ester groups and propiolate ester groups.

In some embodiments, which can be used in conjunction with the above aspects and embodiments, the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or PNIPAAM.

In some embodiments, which can be used in conjunction with the above aspects and embodiments, the unsaturated end groups are linked to the hydrophilic polymer arms by hydrolysable ester groups. For example, the hydrolysable ester groups may be selected from glutarate ester groups, succinate ester groups, carbonate ester groups, or adipate ester groups, among others.

In some embodiments, which can be used in conjunction with the above aspects and embodiments, the system comprises a first precursor composition that comprises the iodinated polyamino compound, a second precursor composition that comprises the reactive multi-arm polymer, and an optional accelerant composition. In some of these embodiments, the first precursor composition is provided in a syringe barrel, the second precursor composition is provided in a vial, and the accelerant composition is provided in a syringe barrel.

In some embodiments, which can be used in conjunction with the above aspects and embodiments, the system further comprises a delivery device.

In other aspects, the present disclosure pertains to medical hydrogels formed by crosslinking the iodinated polyamino compound and the reactive multi-arm polymer of the system of any of the above aspects and embodiments under conditions such that the medical hydrogel is formed. For example, the medical hydrogel may be a medical implant or a medical device coating, among other possibilities.

In other aspects, the present disclosure pertains to methods of treatment comprising administering to a subject a mixture that comprises an iodinated polyamino compound and a reactive multi-arm polymer that comprises a plurality of hydrophilic polymer arms having unsaturated end groups that are reactive with amino groups of the iodinated polyamino compound, for example, in accordance with the any of above aspects and embodiments, under conditions such that the iodinated polyamino compound and the reactive multi-arm polymer crosslink after administration.

Potential benefits associated with the present disclosure include one or more of the following: radiocontrast is maintained, complexity and cost of the manufacturing process is reduced, melting point of the solid components of the hydrogel can be maintained above 40° C. (improving storage and handling), homogeneity of the final hydrogel is improved, in vivo persistence is obtained, and cure kinetics are maintained.

The above and other aspects, embodiments, features and benefits of the present disclosure will be readily apparent from the following detailed description.

### BRIEF DESCRIPTION OF THE DRAWINGS

FIG. 1 schematically illustrates a method whereby a reactive multi-arm polymer, which comprises a core region and a plurality of hydrophilic polymer arms having acrylate end groups is crosslinked with an iodinated polyamino compound, according to an aspect of the present disclosure.

FIGS. 2A, 2B and 2C are chemical drawings of iodinated polyamino compounds, which can be used as crosslinking agents, in accordance with three embodiments of the present disclosure.

FIGS. 3A, 3B and 3C are chemical drawings of iodinated polyamino compounds, which can be used as crosslinking agents, in accordance with three further embodiments of the present disclosure.

FIG. 4 schematically illustrates a method whereby a reactive multi-arm polymer, which comprises a core region and a plurality of polyethylene oxide (PEO) arms having acrylate end groups, is produced by reacting acryloyl chloride with a multi-arm polymer having a core region that comprises a polyol residue and eight hydroxyl-terminated polyethylene oxide arms, according to an aspect of the present disclosure.

FIG. 5 schematically illustrates a method of coupling an iodinated amino acid compound to trilysine to form an iodinated peptide sequence, in accordance with an embodiment of the present disclosure.

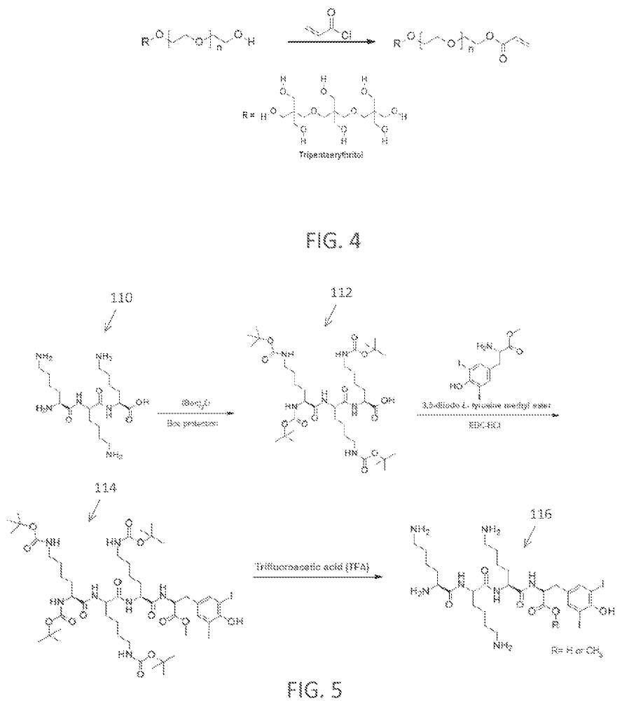

### DETAILED DESCRIPTION

In some aspects of the present disclosure, a radiopaque crosslinked hydrogel is provided that comprises a crosslinked reaction product of (a) an iodinated polyamino compound and (b) a reactive multi-arm polymer that comprises a plurality of unsaturated end groups that are reactive with the amino groups of the iodinated polyamino compound. Unless indicated otherwise, as used herein the prefix “poly” means two or more.

In some aspects of the present disclosure, a system is provided that is configured to dispense an iodinated polyamino compound and a reactive multi-arm polymer that comprises a plurality of unsaturated end groups under conditions such that the iodinated polyamino compound and the reactive multi-arm polymer crosslink with one another. In certain embodiments, those conditions comprise an environment having a basic pH, for example, a pH ranging from about 9 to about 11, typically ranging from about 9.5 to about 10.5, and beneficially ranging from about 9.8 to about 10.2.

In some aspects of the present disclosure, a system is provided that comprises (a) a first composition that comprises an iodinated polyamino compound and (b) a second composition that comprises a reactive multi-arm polymer that comprises a plurality of unsaturated end groups that are reactive with the amino groups of the iodinated polyamino compound. In some embodiments, a third composition in the form of an accelerant composition is provided.

Such a system is advantageous, for example, in that iodine functionality, and thus radiopacity, is provided by the iodinated polyamino compound that acts as a crosslinker for the multi-arm polymer. This allows reactive end groups to be provided on each of the polymer arms, thereby maximizing the crosslinking capacity of the multi-arm polymer, without sacrificing radiopacity.

In various embodiments, the unsaturated end groups of the reactive multi-arm polymer and the amino groups of the iodinated polyamino compound react with one another via Michael addition. In certain embodiments, reaction between the unsaturated end groups and the amino groups is conducted at slightly basic pH (e.g., having a pH value ranging from 7.4 to 11) where the amino groups of the iodinated polyamino compound are deprotonated/neutrally charged and the Michael addition can occur spontaneously at body temperature.

Reactive multi-arm polymers for use herein include those that comprise a plurality of polymer arms (e.g., having two, three, four, five, six, seven, eight, nine, ten, eleven, twelve or more arms), wherein two or more polymer arms of the multi-arm polymers each comprise one or more unsaturated end groups. Examples of unsaturated end groups include unsaturated end groups having double carbon-carbon bonds such as acrylate ester groups,

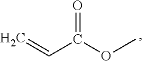

and unsaturated end groups having triple carbon-carbon bonds such as propiolate ester groups,

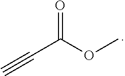

In some embodiments, compositions containing the reactive multi-arm polymers may be provided in which a percentage of the polymer arms comprising one or more unsaturated end groups may correspond to between 50% and 100% of the total number of polymer arms in the composition (e.g., ranging anywhere from 50% to 70% to 80% to 90% to 95% to 99% to 100% of the total number of polymer arms). Typical average molecular weights for the reactive multi-arm polymers for use herein range from 10 to 50 kDa, among other values. In various embodiments, the reactive multi-arm polymers for use herein have a melting point of 40° C. or greater, preferably 45° C. or greater.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO) (also referred to as polyethylene glycol or PEG), poly(propylene oxide) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), polyhydroxyethyl acrylate, polyhydroxyethyl methacrylate, PEG methyl ether acrylate or PEG methyl ether methacrylate, or PNIPAAM, polysaccharides, and combinations thereof.

In some embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In certain beneficial embodiments, the core region comprises a residue of a polyol that contains two, three, four, five, six, seven, eight, nine, ten or more hydroxyl groups. In certain beneficial embodiments, the core region comprises a residue of a polyol that is an oligomer of a sugar alcohol such as glycerol, mannitol, sorbitol, inositol, xylitol, or erythritol, among others.

In certain embodiments, the unsaturated end groups are linked to the polymer arms via hydrolysable ester groups. Hydrolysable ester groups may be selected, for example, from glutarate ester groups, succinate ester groups, carbonate ester groups, or adipate ester groups.

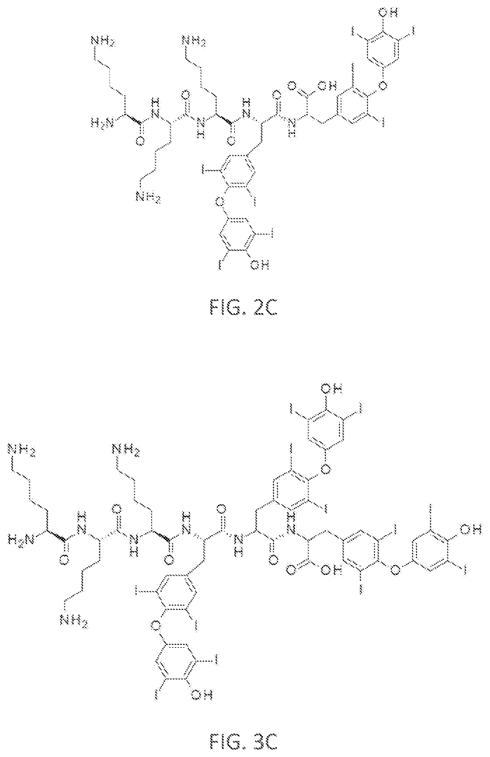

Multi-arm polymers having arms that comprise one or more unsaturated end groups (e.g., acrylate ester groups, propiolate ester groups, etc.) can be formed from multi-arm polymers having arms that comprise one or more hydroxyl end groups. In specific one embodiment shown in FIG. 4, a reactive multi-arm polymer, which comprises a core region and a plurality of polyethylene oxide (PEO) arms having reactive acrylate end groups is formed by reacting acryloyl chloride with hydroxyl end groups of a multi-arm polymer having a core region that comprises a polyol residue R and n hydroxyl-terminated polyethylene oxide arms where n ranges from 30 to 140. In particular, an eight-arm commercially available hydroxyl-terminated polymer where R is a tripentaerythritol polyol residue is reacted with acryloyl chloride to create an acrylate-terminated 8-arm PEO. Acrylate-terminated 8-arm PEO (also referred to acrylate-terminated 8-arm PEG) having a tripentaerythritol residue core and acrylate-terminated 4-arm PEO (also referred to an acrylate-terminated 4-arm PEG) having a pentaerythritol residue core are also available from JenKem Technology USA (Plano, Texas, USA).

As previously noted, in various embodiments of the present disclosure, the unsaturated end groups (e.g., acrylate ester groups, propiolate ester groups, etc.) of reactive multi-arm polymers such as those described above can be reacted with amino groups of an iodinated polyamino compound via Michael addition to form a cross-linked composition.

In various embodiments, the iodinated polyamino compounds for use in the present disclosure comprise a polyamino moiety that is linked to a carboxy-substituted iodinated moiety. In certain embodiments, the polyamino moiety is linked to the carboxy-substituted iodinated moiety through an amide group. In particular embodiments, detailed below, the iodinated polyamino compounds may comprise peptide oligomers that contain from 2 to 10 lysine and/or ornithine amino acid residues and one or more iodinated amino acid residues.

Carboxy-substituted iodinated moieties of the present disclosure include carboxy-substituted iodinated moieties that comprise an iodinated group and a carboxylic acid or carboxylate group. Carboxylate groups include anionic carboxylate groups, carboxylate amide groups, and carboxylate ester groups.

In some embodiments, the carboxy-substituted iodinated moieties of the present disclosure comprise an iodinated aromatic group (also referred to herein as an iodo-aromatic group) and a carboxylic acid or carboxylate group. Examples of iodinated aromatic groups include iodine-substituted monocyclic aromatic groups and iodine-substituted multicyclic aromatic groups, such as iodo-phenyl groups and iodo-naphthyl groups. The aromatic groups may be substituted with one two, three, four, five, six or more iodine atoms.

In particular embodiments, the carboxy-substituted iodinated moieties of the present disclosure comprise at least one hydroxy-iodo-aromatic group and a carboxylic acid or carboxylate group. Examples of hydroxy-iodo-aromatic groups include hydroxy-iodo-phenyl groups and hydroxy-iodo-naphthyl groups. More particular examples of hydroxy-iodo-aromatic groups include hydroxy-iodo-phenyl groups selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, a tri-hydroxy-di-iodo-phenyl group.

In some embodiments, the carboxy-substituted iodinated moieties of the present disclosure comprise iodinated amino acid residues. As used herein, an “amino acid” is an organic compound that contain an amino group (—NH₂), a carboxylic acid group (—COOH), and a side group that is specific to each amino acid. Depending on the surrounding pH, the amino group may be positively charged (—NH₃⁺) and/or the carboxylic acid group may be negatively charged (—COO⁻). An iodinated amino acid is an amino acid in which the side group contains one or more iodine atoms.

Examples of iodinated amino acid residues include iodinated alpha-amino acid residues, iodinated beta-amino acid residues, iodinated gamma-amino acid residues and iodinated delta-amino acid residues.

Examples of iodinated amino acid residues include amino acid residues that comprise an iodinated aromatic group. As previously noted, examples of iodinated aromatic groups include iodo-phenyl groups and iodo-naphthyl groups. In particular embodiments, the iodinated amino acid residues include amino acid residues that comprise a hydroxy-iodo-aromatic group, such as a hydroxy-iodo-phenyl group or a hydroxy-iodo-naphthyl group. More particular examples of hydroxy-iodo-aromatic groups include hydroxy-iodo-phenyl groups selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, a tri-hydroxy-di-iodo-phenyl group, as previously indicated.

Specific examples of iodinated amino acid residues include residues of the following iodinated amino acids: iodo-phenylalanine,

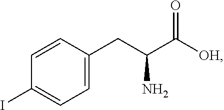 [SMILES: N[C@@H](Cc1ccc(I)cc1)C(=O)O]

which comprises a mono-iodo-phenyl group, monoiodotyrosine,

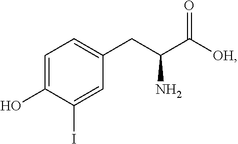 [SMILES: N[C@@H](Cc1ccc(O)c(I)c1)C(=O)O]

which comprises a mono-iodo-phenyl group, specifically, a mono-hydroxy-mono-iodo-phenyl group, diiodotyrosine,

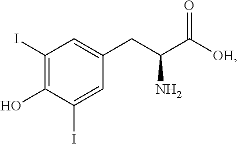 [SMILES: N[C@@H](Cc1cc(I)c(O)c(I)c1)C(=O)O]

which comprises a mono-hydroxy-di-iodo-phenyl group, diiodothyronine,

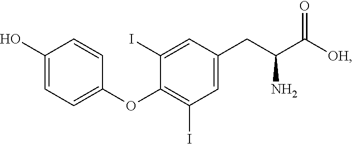 [SMILES: N[C@@H](Cc1cc(I)c(Oc2ccc(O)cc2)c(I)c1)C(=O)O]

which comprises a di-iodo-phenyl group and a hydroxy-phenyl group, triiodothyronine also known as T3,

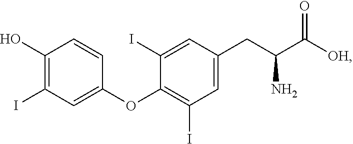 [SMILES: N[C@@H](Cc1cc(I)c(Oc2ccc(O)c(I)c2)c(I)c1)C(=O)O]

which comprises a di-iodo-phenyl group and a mono-hydroxy-mono-iodo-phenyl group, tetraiodothyronine also known as thyroxine or T4,

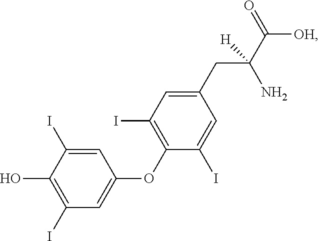 [SMILES: N[C@@H](Cc1cc(I)c(Oc2cc(I)c(O)c(I)c2)c(I)c1)C(=O)O]

which comprises a di-iodo-phenyl group and a mono-hydroxy-di-iodo-phenyl group, iodo-phenylalanine, and 6-iodo-L-DOPA, which comprises a di-hydroxy-mono-iodo-phenyl group, among others. Many of these iodinated amino acids are relatively water soluble, and some, including 3,5-diiodotyrosine and L-thyroxine, are well-studied as monomers for bioerodible polymers.

As described further below, the iodinated polyamino compounds of the present disclosure may be formed by amide coupling reaction between a carboxyl-substituted polyamino compound, selected for example, from those described below (after protection of the amino groups) and an iodinated amino acid derivative, for example, a C₁-C₅-alkyl ester of an iodinated amino acid, preferably a methyl ester of an iodinated amino acid, which effectively acts as a protective group for the carboxylic acid group of the final iodinated polyamino compound. Examples of such iodinated amino acid derivatives include C₁-C₅-alkyl esters of any of the preceding iodinated amino acids. After coupling, the protective groups on the residue of the carboxyl-substituted polyamino compound are removed, and the C₁-C₅-alkyl ester may be converted into the corresponding carboxylic acid or anionic carboxylate group, thereby providing the final iodinated polyamino compound.

In addition to a carboxy-substituted iodinated moiety, such as one of those described above, the iodinated polyamino compounds of the present disclosure also comprise a polyamino moiety linked to the carboxy-substituted iodinated moiety.

In various embodiments, the iodinated polyamino compounds of the present disclosure comprise a polyamino moiety having a plurality (two, three, four, five, six, seven, eight, nine, ten or more) amino groups. For example, the polyamino moiety may comprises a plurality of (two, three, four, five, six, seven, eight, nine, ten or more) —(CH₂)ₓ—NH₂ groups where xis 0, 1, 2 3, 4, 5 or 6. In some of these embodiments, the polyamino moiety may comprises a plurality of —(CH₂)ₓ—NH₂ groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, such as a peptide moiety, a polyalkylene moiety, or a polysaccharide moiety, among others.

In some embodiments, the polyamino moiety of the iodinated polyamino compounds may correspond to a residue of a carboxyl-substituted polyamino compound (a compound comprising a carboxyl group and a plurality of amino groups). Examples of carboxyl-substituted polyamino compounds include peptides containing from 2 to 10 lysine and/or ornithine amino acid residues, including polylysines (e.g., dilysine, trilysine, tetralysine, pentalysine, etc.) and carboxyl-terminated polyamines such as carboxyl-terminated poly(allyl amine), carboxyl-terminated poly(vinyl amine), or carboxyl-terminated chitosan.

Commercially available examples of carboxyl-substituted polyamino compounds also include 16-amino-3-[2-[(4-aminobutyl)(3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N²-[1-[N²-[N²-(N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-ornithyl-L-lysine, D-lysyl-D-lysyl-D-lysine, glycylglycyl-L-lysylglycylglycyl-L-lysine, N²-[N⁴-[N-[N-(N-glycylglycyl)glycyl]glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine, L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N2-(N2-L-ornithyl-L-lysyl)-L-lysine, lysyllysyl-lysine, and L-lysyl-L-lysyl-L-lysyl-L-alanine.

As previously noted, in certain embodiments, the iodinated polyamino compounds of the present disclosure comprise a polyamino moiety linked to a carboxy-substituted iodinated moiety through an amide group. The amide group may be the result of a coupling reaction between an amino group of an iodinated amino acid such as one of those described above and a carboxyl group of a carboxyl-substituted polyamino compound, such as one of those described above. The resulting iodinated polyamino compounds therefore comprise a residue of the carboxyl-substituted polyamino compound and an iodinated amino acid residue. Stated another way, the iodinated polyamino compounds of the present disclosure may be formed by an amidation reaction in which the carboxyl group of a carboxyl-substituted polyamino compound is reacted with an amino group of the iodinated amino acid to form an amide bond between the two residues.

In some aspects, the present disclosure pertains to processes of making iodinated polyamino compounds such as those described above.

In a first process step, amino groups of a carboxyl-substituted polyamino compound may be protected with a suitable protective agent. The amino groups are protected for compatibility with other reactants in a subsequent amide coupling reaction (described below). For example, amino groups of a carboxyl-substituted polyamino compound may be protected by reaction with di-tert-butyl dicarbonate.

In a particular example, and with reference to FIG. 4, amino groups of trilysine (110) are protected using di-cert-butyl dicarbonate (CAS #24424-99-5), thereby forming tBoc-protected trilysine (112). This leaves the carboxyl group of the protected compound (tBoc-protected trilysine) available for amide coupling. In a second step, an iodinated amino acid derivative, specifically an iodinated amino acid C₁-C₅-alkyl ester, is coupled with the protected carboxyl-substituted polyamino compound formed in the first process step in an amide coupling reaction (e.g., via a carbodiimide coupling reagent) to form a protected iodinated polyamino compound. In a particular example, and with reference to FIG. 4, the tBoc-protected trilysine (112) is coupled to an iodinated amino acid derivative (specifically 3,5-diiodo-L-tyrosine methyl ester (CAS #76318-50-8)) in the presence of 1-ethyl-3-(3-dimethylaminopropyl)carbodiimide hydrochloride (EDC·HCl) in dimethylformamide (DMF), yielding a t-Boc protected iodinated peptide oligomer (t-Boc-Lys-Lys-Lys-Tyr-I2) (114). 3,5-diiodo-L-tyrosine has been used to improve radiopacity in in vivo biological applications (see, e.g., U.S. Patent App. No. US 2019/0142863A1 and U.S. Pat. No. 8,288,505). In a third process step, deprotection and hydrolysis of the product of the second process step is performed (e.g., under acidic conditions), to form a final carboxyl-substituted iodinated polyamino compound. For example, as shown in FIG. 4, the t-Boc protected iodinated peptide segment (t-Boc-Lys-Lys-Lys-Tyr-I2) (114) is deprotected and hydrolyzed under acidic conditions using an acid such as trifluoroacetic acid to form an activated iodinated polyamino compound (Lys-Lys-Lys-Tyr-I2) (116).

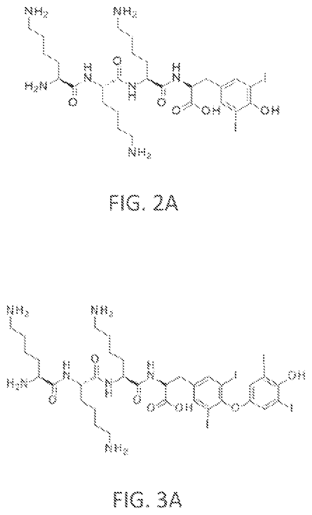

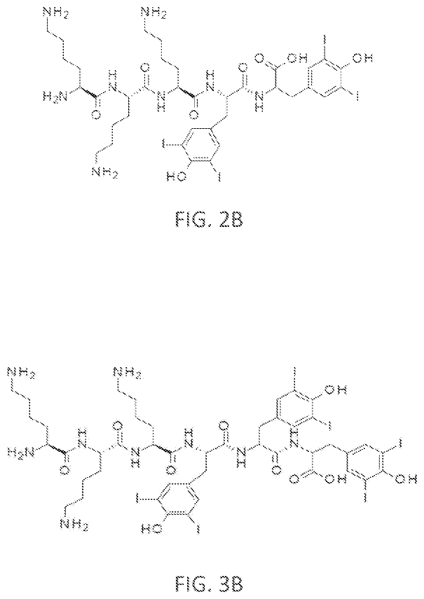

The preceding process can be performed using a variety of carboxyl-substituted polyamino compounds and a variety of iodinated amino acid derivatives. With regard to the latter, when a diiodotyrosine methyl ester is coupled to trilysine as described above, the resulting compound is that shown in FIG. 2A, whereas when thyroxine methyl ester is coupled to trilysine, the resulting compound is that shown in FIG. 3A.

Moreover, more iodinated amino acid groups can be successively added to the chain end, by repeating the first, second and third processes, except that the iodinated polyamino compound formed in the third process is substituted for the trilysine in the first process step, forming a protected compound, which is then coupled to another an iodinated amino acid C₁-C₅-alkyl ester along the lines of the second process in an amide coupling reaction, followed by deprotection and hydrolysis along the lines of the third process. The result of performing these additional steps once, where the iodinated amino acid C₁-C₅-alkyl ester is diiodotyrosine methyl ester is shown in FIG. 2B, which contains two diiodotyrosine residues. The result of performing these additional steps once where the iodinated amino acid C₁-C₅-alkyl ester is thyroxine methyl ester is shown in FIG. 3B, which contains two thyroxine residues.

If the first, second and third processes are repeated again, with the product of FIG. 2B substituted for the trilysine and with diiodotyrosine methyl ester being used as the iodinated amino acid C₁-C₅-alkyl ester, the result is the product shown in FIG. 2C, which contains three diiodotyrosine residues. If the first, second and third processes are repeated, with the product of FIG. 3B substituted for the trilysine and with thyroxine methyl ester being used as the iodinated amino acid C₁-C₅-alkyl ester, the result is the product shown in FIG. 3C, which contains three thyroxine residues. Additional iodinated amino acid residues can be added as desired.

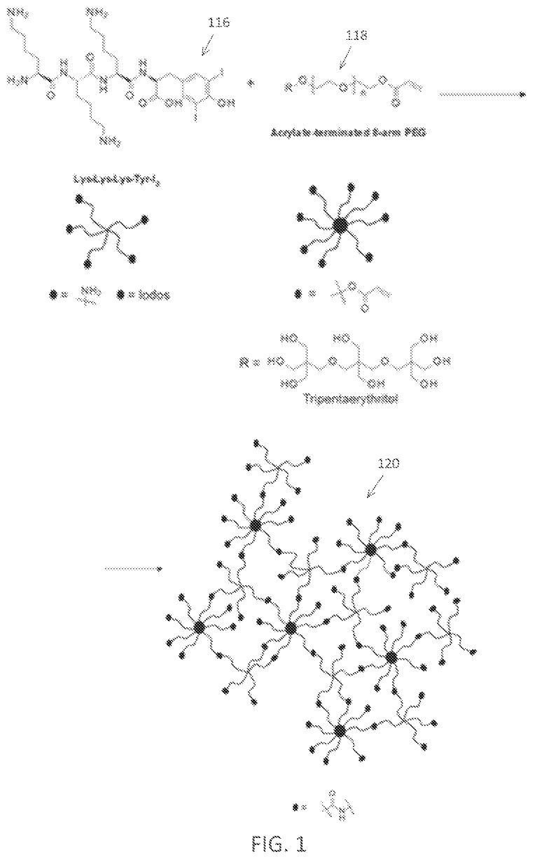

As noted above, in some aspects of the present disclosure, a radiopaque crosslinked hydrogel is provided that comprises a crosslinked reaction product of (a) an iodinated polyamino compound, such as those described above, and (b) a reactive multi-arm polymer that comprises a plurality of polymer arms that have unsaturated end groups, such as those described above, which are reactive with the amino groups of the iodinated polyamino compound via Michael addition. In an embodiment of the present disclosure, and with reference to FIG. 1, an iodinated polyamino compound, specifically, an iodinated peptide oligomer (Lys-Lys-Lys-Tyr-I2) (116) and an acrylate-terminated 8-arm PEG having a pentaerythritol residue core (118) are combined under conditions such that Michael addition occurs between the acrylate groups of the acrylate-terminated 8-arm PEG 118 and the amino groups of the iodinated peptide oligomer (116) thereby forming a crosslinked hydrogel 120. For example, the Michael addition can proceed in aqueous solution under slightly basic conditions (e.g., at a pH ranging from 7.4 to 11 in the presence of buffer solution such as phosphate-buffer saline (PBS) or a borax-related buffer solution, among others.

In various embodiments, the crosslinked hydrogels of the present disclosure are visible under fluoroscopy. In various embodiments, such crosslinked products have a radiopacity that is greater than 250 Hounsfield units (HU), beneficially anywhere ranging from 250 HU to 500 HU to 750 HU to 1000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In various embodiments, the unsaturated end groups of the reactive multi-arm polymer and the amino groups of the iodinated polyamino compound react with one another form a crosslinked product.

In some aspects of the present disclosure, systems are provided that are configured to deliver an iodinated polyamino compound and a reactive multi-arm polymer that comprises a plurality of unsaturated end groups that are reactive with the amino groups of the iodinated polyamino compound under conditions such that the iodinated polyamino compound and the reactive multi-arm polymer crosslink with one another. Such systems can be used to form crosslinked hydrogels, either in vivo or ex vivo.

For example, and again with reference to FIG. 1, a second composition comprising a reactive multi-arm polymer (118) like that described above, which comprises a core region and a plurality of hydrophilic polyethylene oxide arms having unsaturated end groups (i.e., acylate end groups) (where R is a core region, such as a tripentaerythritol polyol residue, and n is eight) can be crosslinked with a first composition comprising an iodinated polyamino compound (116) like that described above, which comprises amino groups that can be reacted with the unsaturated end groups of the reactive multi-arm polymer (118), to form a crosslinked product (120), which may be in the form of a hydrogel when hydrated.

An advantage to this approach is that the iodination is separate from the parent polymer, and the reactive multi-arm polymer with unsaturated end groups can be swapped out with multi-arm polymers having hydrophilic polymer arms other than polyethylene oxide arms and having unsaturated end groups, for example, synthetic, natural, or hybrid synthetic-natural hydrophilic polymer arms such as those described above.

As previously noted, in some aspects of the present disclosure, systems are provided that comprise (a) a first composition that comprises an iodinated polyamino compound and (b) a second composition that comprises a reactive multi-arm polymer that comprises a plurality of unsaturated end groups that are reactive with the amino groups of the iodinated polyamino compound.

The first composition may be a first fluid composition comprising the iodinated polyamino compound or a first dry composition that comprises the iodinated polyamino compound, to which a suitable fluid such as water for injection, saline, etc. can be added to form a first fluid composition. In addition to the iodinated polyamino compound, the first composition may further comprise pH adjusting agents and/or additional agents such as those described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise pH adjusting agents and/or additional agents such as those described below.

In some embodiments, the iodinated polyamino compound is initially combined with the unsaturated multi-arm polymer at an acidic pH at which crosslinking between the unsaturated groups of the reactive multi-arm polymer and the amino groups of the iodinated polyamino compound is suppressed. Then, when crosslinking is desired, a pH of the mixture of the iodinated polyamino compound and the reactive multi-arm polymer is changed from an acidic pH to a basic pH, leading to crosslinking between same.

In particular embodiments, the system comprises (a) a first precursor composition that comprises an iodinated polyamino compound as described hereinabove, (b) a second precursor composition that comprises a reactive multi-arm polymer as described hereinabove, and (c) a third composition, specifically, an accelerant composition, that contains an accelerant that is configured to accelerate crosslinking reaction between the iodinated polyamino compound and the reactive multi-arm polymer.

The first precursor composition may be a first fluid composition comprising the iodinated polyamino compound that is buffered to an acidic pH or a first dry composition that comprises the iodinated polyamino compound and acidic buffering composition, to which a suitable fluid such as water for injection, saline, etc. can be added to form a first fluid composition comprising the iodinated polyamino compound that is buffered to an acidic pH. In some embodiments, for example, the acidic buffering composition may comprise monobasic sodium phosphate, among other possibilities. The first fluid composition comprising the iodinated polyamino compound may have a pH ranging, for example, from about 3 to about 5, typically ranging from about 3.5 to about 4.5, and more typically ranging from about 3.8 to about 4.2. In addition to the iodinated polyamino compound and buffering species, the first precursor composition may further comprise additional agents, such as therapeutic agents and/or further imaging agents (beyond the iodine groups that are present in the iodinated polyamino compound).

The second precursor composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the iodinated polyamino compound that is buffered to an acidic pH. In addition to the reactive multi-arm polymer, the second precursor composition may further comprise additional agents, such as therapeutic agents and/or further imaging agents (beyond the iodine groups that are present in the iodinated polyamino compound).

In a particularly beneficial embodiment, the first precursor composition is a first fluid composition comprising the iodinated polyamino compound that is buffered to an acidic pH and the second precursor composition comprises a dry composition that comprises the reactive multi-arm polymer. The first precursor composition may then be mixed with the second precursor composition to provide a prepared fluid composition that is buffered to an acidic pH and comprises both the iodinated polyamino compound and the reactive multi-arm polymer. In a particular example, a syringe may be provided that contains a first fluid composition comprising the iodinated polyamino compound that is buffered to an acidic pH, and a vial may be provided that comprises a dry composition (e.g., a powder) that comprises the reactive multi-arm polymer. The syringe may then be used to inject the first fluid composition into the vial containing the reactive multi-arm polymer to form a prepared fluid composition that contains the iodinated polyamino compound and the reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, such as therapeutic agents and/or further imaging agents (beyond the iodine groups that are present in the iodinated polyamino compound). In a particular example, a syringe may be provided that contains a fluid accelerant composition.

A prepared fluid composition that is buffered to an acidic pH and comprises the iodinated polyamino compound and the reactive multi-arm polymer as described above, and a fluid accelerant composition that is buffered to basic pH, as described above, may be combined form crosslinked hydrogels, either in vivo or ex vivo.

Examples of further imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Ge^{(III)}, Mn^{(III)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, a system is provided that include one or more delivery devices for delivering first and second compositions to a subject.

In some embodiments, the system may include a delivery device that comprises a first reservoir that contains a first composition that comprises an iodinated polyamino compound as described above and a second reservoir that contains a second composition that comprises a reactive multi-arm polymer that comprises a plurality of unsaturated end groups as described above. In some embodiments, the system may include a delivery device that comprises a first reservoir that contains a first composition that comprises the iodinated polyamino compound and the reactive multi-arm polymer and is buffered to an acidic pH, such as the prepared fluid composition previously described, and a second reservoir that contains a second composition, such as the fluid accelerant composition described above. In either case, during operation, the first composition and the second composition are dispensed from the first and second reservoirs and combined, whereupon the iodinated polyamino compound and the reactive multi-arm polymer and crosslink with one another to form a hydrogel.

In particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

Regardless of the first and second compositions selected, in some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

### EXAMPLE

An iodinated polyamino compound, for example, one of those described hereinabove is dissolved in acidic buffer solution (pH value between 3.8-4.2) and then further mixed with reactive star PEG having unsaturated end groups, for example, a star PEG having a polyol residue core region and 8 hydrophilic polyethylene oxide arms having acrylate end groups, as a prepared fluid composition in one syringe. Another buffer solution is prepared having a controlled pH value around 9.8-10.4 as a fluid accelerant composition in another syringe. Subsequently, a hydrogel is formed by combining the prepared fluid composition and the fluid accelerant composition to form crosslinked hydrogel simultaneously.

1. A system for forming a hydrogel that comprises (a) an iodinated polyamino compound and (b) a reactive multi-arm polymer that comprises a plurality of hydrophilic polymer arms having unsaturated end groups that are reactive with amino groups of the iodinated polyamino compound.

2. The system of claim 1, wherein the iodinated polyamino compound comprises a polyamino moiety that is linked to a carboxy-substituted iodinated moiety by an amide group.

3. The system of claim 2, wherein the carboxy-substituted iodinated moiety comprises an iodinated group and a carboxylic acid or carboxylate group.

4. The system of claim 2, wherein the carboxy-substituted iodinated moiety is an iodinated amino acid residue.

5. The system of claim 4, where the iodinated amino acid residue comprises an iodinated aromatic group.

6. The system of claim 2, wherein the carboxy-substituted iodinated moiety comprises an iodinated aromatic group and a carboxylic acid or carboxylate group.

7. The system of claim 5, wherein the iodinated aromatic group is a monocyclic or multicyclic aromatic moiety that is substituted with one or more iodine groups and one or more hydroxyl groups.

8. The system of claim 2, wherein the polyamino moiety comprises a plurality of —(CH₂)ₓ—NH₂ groups where x is 0, 1, 2 3, 4, 5 or 6.

9. The system of claim 8, wherein the plurality of —(CH₂)ₓ—NH₂ groups are disposed along a polymeric moiety.

10. The system of claim 2, wherein the polyamino moiety comprises a residue of a carboxyl-substituted polyamino compound.

11. The system of claim 2, wherein the polyamino moiety comprises two or more amino acid residues selected from residues of lysine, ornithine, and combinations thereof.

12. The system of claim 1, wherein the unsaturated end groups are selected from acrylate ester groups and propiolate ester groups.

13. The system of claim 1, wherein the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or PNIPAAM.

14. The system of claim 1, wherein the unsaturated end groups are linked to the hydrophilic polymer arms by hydrolysable ester groups.

15. The system of claim 14 wherein the hydrolysable ester groups are selected from glutarate ester groups, succinate ester groups, carbonate ester groups, or adipate ester groups.

16. The system of claim 1, wherein the system comprises a first precursor composition that comprises the iodinated polyamino compound, a second precursor composition that comprises the reactive multi-arm polymer, and an optional accelerant composition.

17. The system of claim 16, wherein the first precursor composition is provided in a syringe barrel, the second precursor composition is provided in a vial, and the accelerant composition is provided in a syringe barrel.

18. A medical hydrogel formed by crosslinking an iodinated polyamino compound and a reactive multi-arm polymer that comprises a plurality of hydrophilic polymer arms having unsaturated end groups that are reactive with amino groups of the iodinated polyamino compound under conditions such that the medical hydrogel is formed.

19. The method of claim 18, wherein said medical hydrogel is a medical implant.

20. A method of treatment comprising administering to a subject a mixture that comprises an iodinated polyamino compound and a reactive multi-arm polymer that comprises a plurality of hydrophilic polymer arms having unsaturated end groups that are reactive with amino groups of the iodinated polyamino compound under conditions such that the iodinated polyamino compound and the reactive multi-arm polymer cross-link after administration.
